(19) United States
(12) Patent Application Publication
(10) Pub. No.: US 2025/0197909 A1
(43) Pub. Date: Jun. 19, 2025

(54) SUBMERGED FERMENTATION PROCESS

(71) Applicant: Novozymes A/S, Bagsvaerd (DK)

(72) Inventors: Jonathan Pham, West Sacramento, CA (US); Xianpeng Cai, Mahomet, IL (US); Ronald Mullikin, Sacramento, CA (US); Sjef Cornelissen, Den Haag (NL); Casper Kanstrup Hermansen, Den Haag (NL); Anne Gay Breuner, Valby (DK); Kim Hansen, Klampenborg (DK); Audrey Diano, Davis, CA (US); Brett McBrayer, Sacramento, CA (US); Stephen Brown, Davis, CA (US); Tyler Chandler, Woodland, CA (US); Haiyan Ge, Davis, CA (US); Frederik Kier Riisgaard, Kalundborg (DK); Benny Cassells, Limhamn (SE); Kirstine Jessen Kristensen, Kalundborg (DK); Valentin Sereda, Raleigh, NC (US)

(73) Assignee: Novozymes A/S, Bagsvaerd (DK)

(21) Appl. No.: 17/915,652

(22) PCT Filed: Mar. 30, 2021
(86) PCT No.: PCT/US2021/024816

§ 371 Date: Sep. 29, 2022

Related U.S. Application Data
(60) Provisional application No. 63/005,142, filed on Apr. 3, 2020.

(51) Int. Cl. C12P 21/00; C12N 9/16; C12N 9/36; C12R 1/07; C12R 1/225; C12R 1/66; C12R 1/80; C12R 1/885

(52) U.S. Cl. CPC C12P 21/00; C12N 9/16; C12N 9/2462; C12Y 301/04001; C12Y 302/01017; C12R 2001/07; C12R 2001/225; C12R 2001/66; C12R 2001/80; C12R 2001/885

(57) ABSTRACT

The present invention provides submerged fermentation processes of producing one or more polypeptide of interest, the process comprising: a) fermenting one or more microorganism that produces the one more polypeptide of interest in a fermentation broth, and b) adding at least one enzyme to the fermentation broth: OR co-expressing the at least one enzyme in the one or more microorganism and secreting the at least one enzyme into the fermentation broth in an amount sufficient to reduce the viscosity of the fermentation broth, compared to when the at least one enzyme is not added or co-expressed; and, optionally, c) recovering the one or more polypeptide of interest.

### REFERENCE TO SEQUENCE LISTING

This application contains a Sequence Listing in computer readable form. The computer readable form is incorporated herein by reference.

### FIELD OF THE INVENTION

The present invention relates to submerged fermentation processes of producing a polypeptide of interest.

### BACKGROUND OF THE INVENTION

In submerged fermentations, the viscosity of the fermentation is of significant importance. With increased viscosity, it becomes more challenging to maintain proper mixing of the broth in the fermenter. Imperfect mixing can affect cell growth and lead to lower product formation rates. Furthermore, in aerobic fermentations, a higher agitation speed or agitation rate is required to maintain the same oxygen transfer rate, when the viscosity increases. Often commercial fermentation plants are operated close to the maximum agitation speed/agitation rate, meaning that an increased viscosity provides for a decreased oxygen transfer rate, which will have an impact on the growth and/or production/productivity. There is therefore a desire to control, in particular to reduce the viscosity of a fermentation broth.

### SUMMARY OF THE INVENTION

A high viscosity of the fermentation liquid leads to a number of challenges that the skilled person needs to solve in order to run a fermentation process optimally and to obtain the maximum productivity and yield. One effect of a high viscosity of the fermentation liquid is that liquid mixing becomes less efficient, which can lead to spatial variations in important fermentation parameters, such as the pH, temperature and substrate concentration. Such spatial inhomogeneities can affect fermentation performance negatively. Secondly, mass transfer rates are reduced at higher liquid viscosities. In aerobic fermentation processes, oxygen is, in general, provided by injecting or sparging air, oxygen or mixtures thereof through the fermenter. A high viscosity is detrimental to a high oxygen transfer from the gas phase to the liquid phase and this may reduce the productivity and yield of a fermentation process. Furthermore, at a high viscosity, carbon dioxide produced by the microorganisms in the fermenter is transferred less efficiently from the broth to the gas phase. High concentrations of carbon dioxide in the fermentation liquid can have a toxic effect on the microorganisms producing the polypeptide of interest and therefore negatively affect productivity and yield.

Surprisingly, the instant inventors found that the addition of at least one enzyme, preferably comprising a muramidase, a chitinase, a glucanase or a phosphodiesterase, more preferably comprising a muramidase and a phosphodiesterase, to a submerged fermentation broth resulted in a reduced viscosity compared to when no enzyme was added. Even though the primary parameter affected by the present invention is the viscosity of the fermentation broth, the skilled person using the present invention may observe a number of benefits such as, improved liquid mixing, reduced spatial variation of important fermentation parameters, improved oxygen uptake, improved carbon dioxide removal, higher productivity and improved yield. As such this invention could allow to keep constant viscosity but decrease power input and/or oxygen supply.

Regarding muramidase addition, the instant inventors have studied different classes of muramidases including GH22, GH24, GH25, GH64, GH71 and GH84. To further study the GH25 family, a phylogenetic tree was built and was used to select enzymes across the tree for maximum diversity. The positive results obtained in the examples below support a high likelihood of finding viscosity reducing enzymes across the GH25 families and also within other GH families.

Accordingly, in a first aspect, the invention relates to submerged fermentation processes of producing one or more polypeptide of interest, the process comprising:
a) fermenting one or more microorganism that produces the one more polypeptide of interest in a fermentation broth, and
b) adding at least one enzyme to the fermentation broth; OR co-expressing the at least one enzyme in the one or more microorganism and secreting the at least one enzyme into the fermentation broth in an amount sufficient to reduce the viscosity of the fermentation broth, compared to when the at least one enzyme is not added or co-expressed; and, optionally,
c) recovering the one or more polypeptide of interest.

### BRIEF DESCRIPTION OF DRAWINGS

FIG. 1. Relative viscosity throughout a fed-batch fermentation of a Bacillus licheniformis strain expressing an amylase product. Relative viscosity is measured for a reference batch without lysozyme added, a batch with lysozyme added continuously through the sucrose feed and a batch with lysozyme added directly as a bolus into the fermenter after 40 hours of fermentation.

### DEFINITIONS

Muramidase: The term “muramidase” activity is defined herein as an O-glycosyl hydrolase, which catalyses the hydrolysis of the glycosidic bond between two or more carbohydrates, or between a carbohydrate and a non-carbohydrate moiety. Muramidases cleave the glycosidic bond between certain residues in mucopolysaccharides and mucopeptides of bacterial cell walls, such as 1,4-beta-linkages between N-acetylmuramic acid and N-acetyl-D-glucosamine residues in a peptidoglycan and between N-acetyl-D-glucosamine residues in chitodextrins, resulting in bacteriolysis. Muramidase belongs to the enzyme class EC 3.2.1.17. This includes enzymes active on the muramidase assay described in materials and methods.

Chitinase: The term “chitinase” activity is defined herein as an O-glycosyl hydrolase that catalyses the hydrolysis of the glycosidic bonds between two or more carbohydrates, or between a carbohydrate and a non-carbohydrate moiety. Chitinases cleaves the glycosidic bond between certain residues in chitins found in fungal cell walls, such as 1,4 beta-linkages between N-acetyl-Glucosamine residues in chitins and chitosan. Endochitinases (EC 3.2.1.14) randomly splits chitin at internal sites of the chitin chain. Exochitinases have also been divided in two categories, Chitobiosidases (EC 3.2.1.29) and beta-1,4-N-acetylglucosaminidases (EC 3.2.1.30). The amino acid sequence of one preferred chitinase is shown in SEQ ID NO:416.

Glucanase: The term “glucanase” activity is defined herein as an O-glycosyl hydrolase that catalyses the hydrolysis of the glycosidic bonds between two or more carbohydrates, or between a carbohydrate and a non-carbohydrate moiety. Glucanases cleaves the glycosidic bond between certain residues in glycans found in fungal cell walls, such as 1,3 beta-linkages, 1,4 beta-linkages or 1,6 beta-linkages between glucose residues in 1,3 glucans, 1,4-glucanse, 1,6-glucans or mixed glucans comprising both 1,3-, 1,4-, and/or 1,6 linkages.

Phosphodiesterase: The term “phosphodiesterase” or “PDE” is defined herein as an enzyme that breaks a phosphodiester bond, typically, in DNA and/or RNA.

Co-cultivation: The term “co-cultivating” is defined herein as a fermentation process where two or more microorganisms are inoculated into the same fermenter and both are cultivated to provide different components of the fermentation broth. In one preferred co-cultivation one microorganism produces the polypeptide of interest and a second microorganism expresses and secreted an enzyme in a sufficient amount to reduce the viscosity of the fermentation broth.

Expression: The term “expression” includes any step involved in the production of a polypeptide including, but not limited to, transcription, post-transcriptional modification, translation, post-translational modification, and secretion.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

### DETAILED DESCRIPTION OF THE INVENTION

The invention relates to submerged fermentation processes of producing one or more polypeptide of interest, the process comprising:
a) fermenting one or more microorganism that produces the one more polypeptide of interest in a fermentation broth, and
b) adding at least one enzyme to the fermentation broth; OR co-expressing the at least one enzyme in the one or more microorganism and secreting the at least one enzyme into the fermentation broth in an amount sufficient to reduce the viscosity of the fermentation broth, compared to when the at least one enzyme is not added or co-expressed; and, optionally,
c) recovering the one or more polypeptide of interest.

In the present description and claims, a submerged fermentation process is intended to mean a fermentation process where one or more microorganisms are grown in a liquid substrate comprising the necessary nutrients, minerals, vitamins and other components necessary for the growth of the one or more microorganisms. Submerged fermentation processes may be aerobic, where air, oxygen or a mixture is added to the fermenter, or it may be anaerobic where no oxygen is added. Typically, submerged fermentation processes are provided with a form for stirring or agitation of the fermentation broth in order to secure uniform conditions in all parts of the fermenter and evenly distribution of all ingredients in the fermentation broth. In aerobic fermentation processes stirring or agitation is usually required in order to secure a good distribution of (small) air bubbles in the fermentation broth which is required for a good oxygen transfer between the gas phase into the liquid phase.

The fermentation process may be a batch process, where all ingredients are added to the fermentation tank that is inoculated with the one or more microorganisms and the fermentation proceeds until completion. In the case of an aerobic fermentation process, oxygen is delivered throughout the fermentation process. The fermentation process may be a fed-batch process, starting as a batch fermentation but a given time, typically when the cell density has reached a certain predetermined level, a nutrient solution (feed) is supplied (fed) to the fermenter until the end of the fermentation process. The fermentation process may be a continuously fed fermentation where nutrients are continuously suppled to the fermenter and product is continuously removed from the fermenter. Such fermentation processes are known in the art and the present invention is not limited to any particular fermentation process.

The fermentation process may be a process where a single microorganism is grown in the fermenter or it may be a co-cultivation process, where two or more microorganisms are inoculated and grown simultaneously during the fermentation process.

In a preferred embodiment, the polypeptide of interest is a single polypeptide or a mixture of two or more polypeptides.

In another preferred embodiment, the polypeptide or the mixture of two or more polypeptides is one or more enzyme, preferably one or more secreted enzyme; even more preferably the polypeptide or the mixture of two or more polypeptides is selected from a hydrolase, isomerase, ligase, lyase, oxidoreductase, and transferase, e.g., an aminopeptidase, amylase, carbohydrase, carboxypeptidase, catalase, cellobiohydrolase, cellulase, chitinase, cutinase, cyclodextrin glycosyltransferase, phosphodiesterase, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, beta-xylosidase.

The polypeptide of interest can be formed by the complete fermentation broth at the end of the fermentation, or it can be a component or a mixture of components that are recovered from the fermentation broth at the end of the fermentation process using any number of recovery and separation processes known in the area, such as filtrations, centrifugation, precipitation, evaporation, distillation etc.

The fermentation substrate is according to the invention intended to mean the nutrient composition wherein the one or more microorganisms are grown in the fermentation process. The fermentation substrate is generally an aqueous solution comprising a mixture of nutrients, vitamins, minerals and other components necessary for the growth of the particular selected microorganisms for the particular fermentation process. As examples of suitable nutrients can be mentioned carbohydrates, such as mono-, di-, oligo- or polysaccharides e.g. glucose, maltose, lactose, xylose, arabinose, dextrins, maltodextrin and starches; amino acids, di-oligo- and polypeptides; lipids e.g. mono-, di- or triglycerides, milk, juices and fractions thereof. Other examples of suitable substrates included hydrolysates of naturally occurring materials, e.g. hydrolysed starch, cellulose or lignocellulosic materials. Further examples of suitable nutrients include stream from industrial processes, such as molasses, sugar been pulp, cereal fractions, corn steep liquor etc. As example of suitable minerals can be mentioned sodium, potassium, calcium, magnesium, iron and ammonium salts with suitable anions such as chloride, carbonate, sulphate, phosphate, and nitrates, and further micronutrients, i.e. components required in small amounts for growth such a cupper, iron, molybdenum, cobalt, zinc and iodine.

The fermentation substrate may be a defined substrate, i.e. a substrate composed of clearly defined components or is may be a complex substrate comprising one or more complex nutrient that can not be clearly defined, such as (hydrolysed) materials, e.g. hydrolysed starch, hydrolysed lignocellulosic material, hydrolysed protein; corn step liquor or molasses.

When the one or more microorganisms are inoculated into the fermentation substrate the microorganisms grow and covert the nutrients into cell material, products, metabolites and waste material forming a fermentation broth. Thus, the term fermentation broth in intended to mean an aqueous composition comprising nutrients, minerals, cells, cellular materials, products, metabolites, waste products etc; provided by the growth of the one or more microorganism(s) in the fermentation substrate.

The one or more microorganisms may in principle be selected among any prokaryotic or eukaryotic organisms, which organisms are capable of growing in a submerged fermenter.

In a preferred embodiment, the one or more microorganism is bacterial; preferably prokaryotic; more preferably Gram positive or Gram negative; even more preferably the one or more microorganism is selected among Gram-positive bacteria, including, Bacillus, Clostridium, Enterococcus, Geobacillus, Lactobacillus, Lactococcus, Oceanobacillus, Staphylococcus, Streptococcus, and Streptomyces; or selected among Gram-negative bacteria, including, Campylobacter, E. coli, Flavobacterium, Fusobacterium, Helicobacter, Ilyobacter, Neisseria, Pseudomonas, Salmonella, and Ureaplasma; most preferably, the one or more microorganism comprises a Bacillus species, preferably selected among Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis and Bacillus thuringiensis.

In another preferred embodiment, the one or more microorganism is a Lactobacillus species, preferably selected among Lactobacillus reuteri, Lactobacillus casei, Lactobacillus paracasei, L. paracasei subsp. paracasei, L. paracasei subsp. tolerans, Lactobacillus rhamnosus, Lactobacillus brevis, Lactobacillus plantarum, Lactobacillus crispatus and Lactobacillus delbrueckii.

The cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The fungal cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolism may be fermentative.

In still another preferred embodiment, the one or more microorganism is eukaryotic, more preferably the one or more microorganism is a fungus, even more preferably a filamentous fungus, including Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma or a yeast, including Candida, Hansenula, Kluyveromyces, Pichia, Saccharomyces, Schizosaccharomyces, Yarrowial, Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica; most preferably the one or more microorganism is an Aspergillus, Penicillium or Trichoderma species, preferably selected from Aspergillus awamori, Aspergillus foetidus, Aspergillus fumigatus, Aspergillus japonicus, Aspergillus nidulans, Aspergillus niger, Aspergillus oryzae, Penicillium purpurogenum, Trichoderma harzianum, Trichoderma koningii, Trichoderma longibrachiatum, Trichoderma reesei and Trichoderma viride.

In a preferred embodiment, the at least one enzyme is added as a bolus or as continuous addition. Alternatively, the process is a fed batch or a continuously fed fermentation process, and the at least one enzyme is added in the feed.

Preferably, the at least one enzyme is selected from muramidase, chitinase, glucanase, mannanase, nuclease and phosphodiesterase; more preferably the at least one enzyme is a muramidase, even more preferably a GH22, GH24, GH25, GH64, GH71 and GH84 glycosyl hydrolase; still more preferably the at least one enzyme comprises a muramidase having an amino acid sequence at least 60% sequence identity; e.g. at least 65% sequence identity; e.g. at least 70% sequence identity; e.g. at least 75% sequence identity; e.g. at least 80% sequence identity; e.g. at least 85% sequence identity; e.g. at least 90% sequence identity; e.g. at least 95% sequence identity; e.g. at least 96% sequence identity; e.g. at least 97% sequence identity; e.g. at least 98% sequence identity; e.g. at least 99% sequence identity; or even 100% sequence identity to the amino acid sequence shown in any of SEQ ID NO: 1 to SEQ ID NO:453.

With respect to dosage of the at least one enzyme, a preferred embodiment comprises adding up to 20,000 mg of the at least one enzyme per kg of fermentation broth; preferably up to 15,000 mg/kg; even more preferably up to 10,000 mg/kg; still more preferably up to 5,000 mg/kg; even more preferably up to 1,000 mg/kg and most preferably up to 500 mg/kg.

Alternatively, in another preferred embodiment, the at least one enzyme is added in an activity ranging from below the detection limit of the muramidase assay described herein and up to an activity of 0,200 limit of the muramidase assay described herein when diluted 4,000 times; preferably when diluted 3,000 times; more preferably up to 0,200 when diluted 2,000 times; even more preferably up to 0,200 when diluted 1,000 times; still more preferably up to 0,200 when diluted 500 times; most preferably up to 0,200 when diluted 100 times, as described in examples 1 and 16 of WO2018/113743—chicken egg white muramidase under this assay should give an OD450 drop of 0.18 at 5 mg/L as per WO2018/113743 example 16; if not the values should be normalized accordingly.

In a preferred embodiment, the at least one enzyme is a chitinase, which is added to the fermentation broth in amounts ranging from 0.1 ppm-500 ppm, particularly from 0.1-100 ppm, more particularly from 0.1-50 ppm, such as from 0.5-10 ppm, calculated as amount of enzyme protein to the amount of fermentation broth.

In a preferred embodiment, the at least one enzyme is a glucanase, which is added to the fermentation broth in amounts ranging from 0.1 ppm-500 ppm, particularly from 0.1-100 ppm, more particularly from 0.1-50 ppm, such as from 0.5-10 ppm, calculated as amount of enzyme protein to the amount of fermentation broth.

A composition comprising chitinases and glucanases may e.g. be provided by fermenting the filamentous fungus Trichoderma harzianum under conditions inducing the production of such enzymes, for example in form of the commercially available enzyme product Glucanex™, manufactured by Novozymes A/S. The Glucanex™ is preferably added in amounts ranging from 0.1 ppm-500 ppm, particularly from 0.1-400 ppm, more particularly from 0.1-300 ppm, even more particularly from 0.1-200 ppm or from 0.1-100 ppm, such as, from 0.5-10 ppm.

Techniques for cloning genes encoding the at least one enzyme are known in the art and the skilled person will be able to select suitable methods and techniques for expressing the relevant genes for use according to the present invention.

In one embodiment the at least one enzyme is added by co-expressing and secreting the at least one enzyme in the one or more microorganism that also produces the polypeptide of interest.

In another embodiment the at least one enzyme is added to the fermentation broth by co-cultivating a second microorganism in the fermentation broth, wherein the second microorganism produces and secretes the at least one enzyme into the fermentation broth.

In a preferred embodiment, the oxygen transfer rate in the fermentation broth is increased by increasing the agitation, stirring speed, mixing or oxygenation of the fermentation broth during the fermentation.

According to the invention, a reduced viscosity is achieved in the fermentation broth during at least part of the fermentation process compared with a similar fermentation process performed with same substrate and same microorganism(s) but without addition of the one or more enzyme.

### EXAMPLES

### Materials and Methods

### Muramidases:

A muramidase having the sequence of SEQ ID NO: 1 was used in the Examples below. This muramidase is identical to the muramidase disclosed in WO 2013/076253 as SEQ ID NO: 4 and further details disclosing how the enzyme can be obtained can be found in that publication.

The inventors fully expect other enzymes, e.g., GH18, GH22, GH24, GH25, GH64, GH71, GH84 glycohydrolases and others as exemplified below and also provided in the enclosed sequence list, to be functional according to the invention; examples thereof are provided in SEQ ID NO:2 to SEQ ID NO:453, where the intention is, of course, that mature versions of the amino acid sequences listed are what is to be added to the fermentation broths.

### Muramidase Assay:

Muramidase activity was determined as described in examples 1 and 16 of WO2018/113743; chicken egg white muramidase under this assay should give an OD450 drop of 0.18 at 5 mg/L as per WO2018/113743 example 16; if not, the values should be normalized accordingly.

### Microorganisms:

Bacillus licheniformis strains used for the experiments in Examples 1, 2 and 3 are shown below in table 1.

TABLE 1

| Strains | Product produced |
|---|---|
| Strain 1 | Amylase |
| Strain 2 | Mannanase |
| Strain 3 | Amylase |
| Strain 4 | Amylase |
| Strain 5 | Protease |
| Strain 6 | Phosphodiesterase |
| Strain 7 | Phytase |

### Example 1. Reducing Culture Broth Viscosity by Adding Muramidase to a Fermentation for Amylase Production with Recombinant Bacillus licheniformis

A Bacillus licheniformis strain producing an amylase product was grown in three identical fermenters, containing a defined mineral medium. The fermentations were fed with a sucrose solution. The feed rate was controlled by the dissolved oxygen level. One fermenter, referred to as the reference served as the experimental control and had no muramidase added to the fermentation broth. The second fermenter was supplied with a bolus of 1.6 mg muramidase/kg start fermentation broth after 40 hours of fermentation. The third fermenter was continuously supplied with muramidase through a separate sucrose feed line. To the separate sucrose feed 3.2 mg muramidase/kg sucrose feed was added. The fermentation was ended after 80 hours.

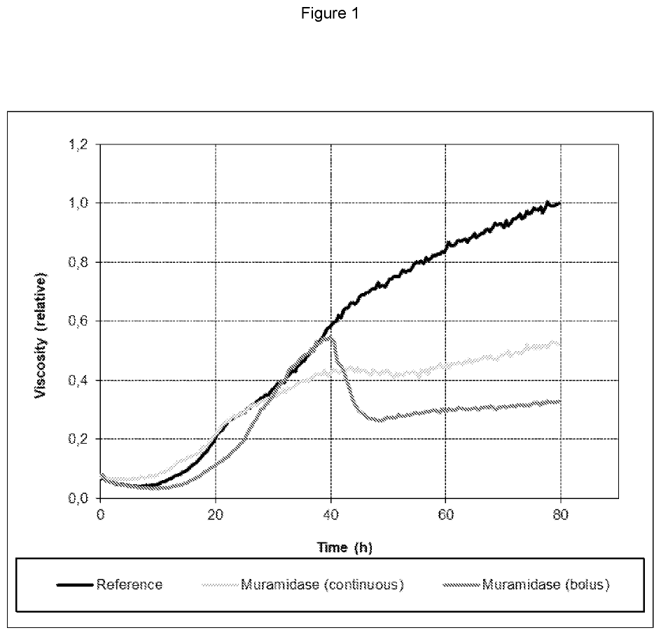

Viscosity was measured on a Hydramotion Inline Viscosimeter XL7 according to the manufacturer's instructions. Viscosity measurements showed that, in the reference fermentation, viscosity increases as the fermentation progresses linked to the production of biomass and/or other fermentation products. The addition of muramidase, supplied either as a bolus or continuously, reduces the viscosity of the Newtonian fermentation broth compared to the reference (FIG. 1). Supplying muramidase continuously results in a more gradual viscosity reduction compared to the reference, whereas a bolus addition shows more instant reduction in viscosity. Both methods of addition are effective to reduce the viscosity of the culture broth compared to the reference.

### Example 2. Adding Muramidase to Fermentations for Amylase or Mannanase Production with Recombinant Bacillus licheniformis Results in Higher Fermentations Yield

Example 1 showed a reduction in viscosity by adding muramidase to a fermentation broth, and this reduction allowed other process modifications resulting in yield improvements.

Multiple Bacillus licheniformis strains (Table 1) producing either an amylase or mannanase product were grown in fermenters; all of the strains were grown in a fed-batch process fed with a sucrose solution and with the feed rate controlled by the dissolved oxygen level.

Strain 1 was supplied with 1.6 mg muramidase/kg start fermentation broth (added as a bolus) after 40 hours of fermentation. Strain 2, 3 and 4 were supplied with 18 mg muramidase/kg start fermentation broth (added as a bolus) after 25 hours of fermentation. The fermentation of Strain 1 was ended after 80 hours, and after 120 hours for Strains 2, 3 and 4.

The results showed that by adding muramidase to the four Bacillus licheniformis strains, the yield increased compared to a reference without muramidase added (Table 2). The term yield describes the total amount of enzyme produced at the end of the fermentation relative to the reference fermentation.

TABLE 2
Yield improvements between a reference batch without muramidase
added and a batch with muramidase added. Strain 1 had 1.6
mg muramidase/kg start fermentation broth added after 40
hours. Strain 2, 3 and 4 had 18 muramidase/kg start fermentation
broth added after 25 hours of fermentation.

Yield

| Strains | Reference | Muramidase |
|---|---|---|
| Strain 1 | 100% | 119% |
| Strain 2 | 100% | 116% |
| Strain 3 | 100% | 116% |
| Strain 4 | 100% | 110% |

### Example 3. Yield Improvement in Fermentations for Amylase Production with Recombinant Bacillus licheniformis Depends on Added Muramidase Concentration

A Bacillus licheniformis strain producing an amylase product (Strain 1) was grown in a fermenter. The fermentation was fed with a sucrose solution. The feed rate was controlled by the dissolved oxygen level. Different concentrations of muramidase (ranging from 0 to 500 mg muramidase/kg start fermentation broth) were added after 25 hours of fermentation as a bolus. The reference batch without muramidase was supplied with comparable volume of water instead of muramidase solution.

After ending the fermentations, the yield was calculated from each fermentation and compared to a reference with no added muramidase. The results found an increase in yield compared to the reference at increasing concentrations of muramidase (Table 3).

TABLE 3
Yield improvements with increasing amounts of muramidase added
to the fermentation broth compared to a reference without
any muramidase addition. 1, 50 and 500 mg muramidase/kg start
fermentation broth were added after 25 hours of fermentation
as a bolus directly in the fermenters.
Muramidase addition (mg muramidase/kg start fermentation broth)

| | 0 | 1 | 50 | 500 |
|---|---|---|---|---|
| Yield | 100% | 126% | 145 ± 4.4% | 154 ± 4.4% |

### Example 4. Viscosity Reduction of Muramidase in Combination with Oxygen Enrichment

A Bacillus licheniformis strain producing a protease product (Strain 5) was fermented in a fed batch process with a complex media fed with a sucrose solution controlled by the dissolved oxygen level. Table 4 below shows the experimental setup. This experiment tested the effect of muramidase addition to fermentations with and without oxygen enrichment against a reference process. The reference batch had no muramidase or oxygen enrichment supplied to the fermentation broth. Oxygen enrichment batches with and without muramidase addition were enriched to an average of 35% oxygen of the total airflow in a 100 hour fermentation. The batches with muramidase addition were supplied with two boluses of 50 mg muramidase/kg start fermentation broth after 0 and 48 hours.

Off-line rheological characterization of the fermentation broth was performed by steady state flow measurements using a “vane-and-cup” geometry ideal for suspension rheology in a controlled strain and stress rheometer (AR-G2, TA Instruments, New Castle, DE). The vane consists of four blades (14 mm W 42 mm H) mounted at right angles, and the cup had a 15 mm radius and contained 28.72 mL fermentation broth. The gap between vane and cup was 4,000 mm. Nine steady state measurements (30 s at each shear rate, where the reported values are averages of the last 15 s measurement at each interval) were made for each sample in the shear rate interval from 10 to 150 s⁻¹.

Comparison of offline viscosity measurements at shear rates of 46/s and 100/s at the end of the fermentation showed an increase in viscosity for the oxygen enriched fermentations compared to the reference (Table 4). The viscosity increase upon enrichment of oxygen can be linked to additional biomass formation and/or other fermentation products as a result of increased sucrose dosing. A reduction in viscosity is observed upon the addition of muramidase to the fermentations with oxygen enrichment. Muramidase addition to fermentations without oxygen enrichment showed a viscosity reduction compared to the reference fermentations. The relative viscosity reduction observed between oxygen enriched fermentations with muramidase addition and oxygen enriched fermentations without muramidase is bigger compared to the relative viscosity reduction between the fermentations with muramidase addition and the reference fermentations without muramidase.

TABLE 4
Relative viscosity of fermentations with muramidase addition
(100 mg muramidase/kg start fermentation broth), oxygen
enrichment, and oxygen enrichment with 100 mg muramidase/kg
start fermentation broth, compared to a reference fermentation
without oxygen enrichment and muramidase.

| | | | Oxygen | |
|---|---|---|---|---|
| | | | enrichment | |
| | | | with 100 mg | 100 mg |
| | | | muramidase/ | muramidase/ |
| | | | kg start | kg start |
| | | Oxygen | fermentation | fermentation |
| | Reference | enrichment | broth | broth |
| | Relative viscosity vs. reference | | | |
| Apparent shear | 100 ± 2% | 375 ± 12% | 136 ± 16% | 83 ± 5% |
| rate = 46/s | | | | |
| Apparent shear | 100 ± 3% | 353 ± 10% | 136 ± 13% | 80 ± 3% |
| rate = 100/s | | | | |

### Example 5. Yield Improvement in Fermentations for Protease Production with Recombinant Bacillus licheniformis Increased with Muramidase and Muramidase Combined with Phosphodiesterase Addition

A Bacillus licheniformis strain producing a protease product (Strain 5) was grown in a fermenter. The fermentation was fed with a sucrose solution. The feed rate was controlled by the dissolved oxygen level.

### Experimental Overview

For experiments with muramidase alone, muramidase in different concentrations (ranging from 30 to 75 mg muramidase/kg start fermentation broth) was added after 0 hours and 48 hours fermentation as a bolus.

For experiments with muramidase combined with phosphodiesterase, muramidase in different concentrations (ranging from 30 to 75 mg muramidase/kg start fermentation broth) was added after 0 hours and 48 hours fermentation as a bolus. 25 mg phosphodiesterase kg start fermentation broth was added after 0 hours and 48 hours fermentation as a bolus.

After ending the fermentations, the yield was calculated from each fermentation and compared to a reference with no added enzyme. The results show an increase in yield compared to the reference at increasing concentrations of muramidase (Table 5).

TABLE 5
Yield improvements with no enzyme addition, muramidase added
alone, and muramidase combined with phosphodiesterase.

| Experiment | Yield | Viscosity reduction |
|---|---|---|
| Reference | 0 | 0 |
| Muramidase addition | + | + |
| Muramidase and phosphodiesterase addition | + | + |

When combined with phosphodiesterase, less muramidase was needed to reach the same increase in yield, as when only muramidase was added. The combination of muramidase with phosphodiesterase resulted in the situation that overall less enzyme was required to achieve the same increase in yield.

### Example 6. Viscosity Reduction in Fermentations for Phosphodiesterase Production with Recombinant Aspergillus oryzae with Mutanase Added

An Aspergillus oryzae strain producing a phosphodiesterase product (Strain 6) was grown in a fermenter. The fermentation was fed with a glucose solution. The feed rate was added with a fixed ramp. Identical process parameters were applied and the effect on the oxygen transfer was studied using the following principles.

The total oxygen transfer in the system was determined from a mass balance over the system. The mass transfer rate of oxygen (OTR) can be described by:

$$OTR = kLa ( {DO}^{*} - DO ) ( Nielsen et al 2003 )$$

Where kLa is the volumetric mass transfer coefficient, DO* is the oxygen concentration in the liquid phase at equilibrium with the gas phase, and DO is the actual oxygen concentration in the liquid phase.

kLa is influenced by the mixing power input, the superficial gas velocity, and the fermentation broth viscosity:

$$k La = C ( P / V ) \hat{} a * v\_g \hat{} b * my \hat{} c ( Garcia - Ochoa and Gomez 2009 )$$

where P is the power input, V is the broth volume, v_g is the superficial gas velocity and my is the viscosity.

By keeping the power and superficial gas velocity constant, we can therefore indirectly observe the development in viscosity by calculating the kLa:

$$k La = OTR / ( {DO}^{*} - DO )$$

In addition to viscosity reduction as seen in table 6, adding mutanase to the fermentation also increased the yield of the fermentation.

TABLE 6
Yield improvement and viscosity reduction with addition of mutanase
(SEQ ID NO: 433) to an Aspergillus oryzae fermentation

| Enzyme added | Yield | Viscosity reduction |
|---|---|---|
| Reference | 0 | 0 |
| 80 mg mutanase/kg (SEQ ID NO: 433) start | + | + |
| fermentation broth added after 48 hours | | |
| fermentation as a bolus. | | |

### Example 7. Lower Agitation Needed and Viscosity Reduction in Fermentations for Phytase Production with Recombinant Aspergillus oryzae with Chitinase Added

An Aspergillus oryzae strain producing a phytase product (Strain 7) was grown in a fermenter. The fermentation was fed with a maltose solution. The feed rate was added with a fixed ramp.

The level of dissolved oxygen in the fermentation broth was controlled by manipulation of the agitation speed measured in rounds per minute (RPM) of the agitator. Addition of the chininase led to lower RPM needed to maintain the same level of dissolved oxygen in the broth. As the total oxygen transfer was equal and all other parameters unchanged, the decrease in RPM is equivalent to a viscosity reduction.

TABLE 7
RPM needed to maintain level of dissolved oxygen and
viscosity reduction with addition of chitinase (SEQ ID
NO: 416) to Aspergillus oryzae fermentation

| | RPM | Viscosity |
|---|---|---|
| Enzyme added | reduction | reduction |
| Reference | 0 | 0 |
| 8 mg chitinase (SEQ ID NO: 416)/kg start | + | + |
| fermentation broth added after 96 hours | | |
| fermentation as a bolus. | | |

### Example 8. Yield Improvement in Fermentations for Amylase Production with Recombinant Bacillus licheniformis Using Diverse Glycosyl Hydrolases and Muramidase-Like Enzymes

A Bacillus licheniformis strain producing an amylase product (strain 1) was grown in a defined mineral medium. The fermentations were fed with a sucrose solution. The feed rate was controlled by the dissolved oxygen level. Eight fermenters, referred to as the reference, served as the experimental control and had no muramidase added to the fermentation broth. The other fermenters were supplied with one to two bolus of 18-50 mg muramidase/L start fermentation broth after 22-72 hours of fermentation. The fermentations were ended after five to six days.

After ending the fermentations, the yield was calculated from each fermentation and compared to a reference with no added enzyme. Yield is equal to the activity measured at the end of the fermentation multiplied by the broth weight at the end of the fermentation.

The results show an increase in yield compared to the reference (Table 8).

TABLE 8
Yield improvements with no enzyme addition
(reference) compared to muramidases

| | Yield | Viscosity |
|---|---|---|
| Enzyme added | Improvement | Decrease |
| Reference | 1.00 | 0 |
| Muramidase (n = 4), | 1.39 +/− 0.05 | + |
| SEQ ID NO: 1 | | |
| Mutanolysin (Sigma | 1.17 | + |
| M9901) | | |
| SEQ ID NO: 395 | 1.25 | + |
| SEQ ID NO: 396 | 1.20 | + |
| SEQ ID NO: 397 | 1.29 | + |
| SEQ ID NO: 398 | 1.09 | + |
| SEQ ID NO: 399 | 1.30 | + |
| SEQ ID NO: 400 | 1.22 | + |
| SEQ ID NO: 401 | 1.24 | + |
| SEQ ID NO: 402 | 1.20 | + |
| SEQ ID NO: 403 | 1.13 | + |
| SEQ ID NO: 404 | 1.11 | + |
| SEQ ID NO: 405 | 1.47 | + |
| SEQ ID NO: 406 | 1.09 | + |
| SEQ ID NO: 407 | 1.25 | + |
| SEQ ID NO: 408 | 1.22 | + |
| SEQ ID NO: 409 | 1.27 | + |
| SEQ ID NO: 410 | 1.35 | + |
| SEQ ID NO: 411 | 1.29 | + |
| SEQ ID NO: 412 | 1.27 | + |
| SEQ ID NO: 413 | 1.39 | + |
| SEQ ID NO: 414 | 1.09 | + |
| (GH84) | | |
| Chicken Eggwhite | 1.17 | + |
| (Sigma) | | |

### Example 9. Construction of the B. licheniformis Co-Expression Strain

### Strain:

MaTa338: Bacillus licheniformis strain producing amylase having a kanamycin marker. Strain 1 is the descendant of MaTa338 after removal of the kanamycin marker.

### Plasmid:

pHyGe867: plasmid with a temperature sensitive origin and erm gene coding for erythromycin resistance. The plasmid contains a phosphate depletion inducible promoter driving a muramidase.

### Strain Construction:

The construction of the Bacillus licheniformis strain co-expressing muramidase was done in a similar manner as described in WO2020229191.

The MaTa338 strain was used as a host strain for transformation with pHyGe867. Correct integration of the muramidase gene leads to the removal of the kanamycin expression cassette. A clone was isolated as both erythromycin, kanamycin sensitive and preserved as HyGe877n7. Correct insertion of the muramidase gene was verified by sequencing.

### Example 10. Co-Expression of Muramidase Decreases Fermentation Viscosity

Strain 1 and HyGe877n7 were fermented side by side in a minimal medium without exogeneous enzyme addition. The fermentations were fed with a sucrose solution. The feed rate was controlled by the dissolved oxygen level. The fermentations were ended after five days. After ending the fermentations, the yield was calculated from each fermentation and compared to a reference with no added enzyme. Yield is equal to the activity measured at the end of the fermentation multiplied by the broth weight at the end of the fermentation.

The results show a decrease in viscosity and an increase in yield when the muramidase is co-expressed (Table 9).

TABLE 9
Yield improvements when muramidase is co-expressed

| Enzyme production | Yield | Viscosity reduction |
|---|---|---|
| Strain 1 - reference | 1.00 | 0 |
| HyGe877n7 - co-express | 1.43 | + |

### Example 11. Viscosity Reduction in Fermentations for Cellulase Production with Trichoderma reseei with a Beta 1-3 Glucanase Added

A Trichoderma reseei strain producing wild type cellulases was on PDA plates for 5-9 days at 28° C. Three 500 ml shake flasks each containing 100 ml of shake flask medium for each strain were inoculated with two plugs from the PDA plates. The shake flasks were incubated at 26° C. for 48 hours on an orbital shaker at 250 rpm. The cultures were used as seeds for larger scale fermentation.

A total of 160 ml of each seed culture was used to inoculate Applikon Biotechnology 3-liter glass jacketed fermentors containing 1.8 liters of fermentation batch medium. The fermenters were maintained at a temperature of 28° C. and pH was controlled using an Applikon control system to a set-point of 4.75+/−0.25. Air was added to the vessels at a rate of 2.0 L/min and the broths were agitated by Rushton impeller rotating at 1100 rpm. Fermentation feed medium composed of dextrose and phosphoric acid was dosed at a rate of 0 to 15 g/hour for a period of 167 hours based on a dissolved oxygen-controlled ramp. Each enzyme was added in one or two boluses at the final concentration of maximum 0.2 g/L broth. Additions were made from day 2 to day 6 of fermentation. Daily samples were taken from each fermentor, centrifuged, and stored at −20° C. After ending the fermentations, total protein assay was run. The yield was calculated from each fermentation and compared to a reference with no added enzyme. Yield is equal to the total protein assay results multiplied by the broth weight at the end of the fermentation.

Total protein assay. Day 7 fermentation samples were desalted and buffer exchanged into 50 mM sodium acetate-100 mM NaCl pH 5.0. After desalting the protein concentration of the enzyme compositions was determined using a Gallery Analyzer (Thermo Scientific). Cultures were diluted appropriately in water. An albumin standard (bovine serum albumin; BSA) was serial diluted to a concentration range of 0.66 mg/ml to 0.087 mg/ml in water. A total of 20 μl of each dilution including standard was transferred to a cuvette containing 200 μl of a bicinchoninic acid (BCA) substrate solution (Pierce BCA Protein Assay Kit; Thermo Scientific) and then incubated at 37° C. for 30 minutes. Upon completion of the incubation the optical density of 540 nm was measured for each sample. Sample concentrations were determined by extrapolation from the generated standard curve.

TABLE 10
Yield improvements when viscosity reducing enzymes
are added to the fermentation broth of Trichoderma
reseei compared to no enzyme addition (reference)

| | | Viscosity |
|---|---|---|
| Enzyme(s) added | Yield | reduction |
| Reference | 1.0 | 0 |
| SEQ ID NO: 415; beta 1-3 glucanase GH64 | 1.5 | + |
| Mutanase (8) | 1.4 | + |
| SEQ ID NO: 415 + SEQ ID NO: 416; | 2.0 | ++ |
| endo-beta-1,4-chitinase GH18_5 | | |

### Example 12. Enzyme Induced Culture Broth Viscosity Reduction. (Aspergillus niger Fermentation

The viscosity of the fermentation broth was measured offline with a Rapid Visco™ Analyser with Thermocline (RVA 4500, Perten Instruments of Australia Pty Limited) according to the manufacturer's instructions. Typical measurements were conducted at 34° C. in a range of shear rates between 30 s⁻¹ and 330 s⁻¹. Apparent viscosity was determined by recording the viscosity value at a shear rate of 160 s⁻¹. Data analysis was performed with the software TCW3 (Perten Instruments). Aliquots of treated and untreated (reference) samples were incubated at 34° C. for 3 hours prior viscosity measurements.

An Aspergillus niger fermentation process was used to investigate the effect of enzyme addition to culture broth. Typical fed-batch fermentation involved glucose solution as carbon source with a feed rate controlled by the dissolved oxygen level. Fermentation broth of a strain roducing a AMG-NAroduct was collected at specified time point(s). Viscosity reduction potential of the specific enzymes (Table XX) was examined in a series of offline measurements. Equimolar amounts of enzymes were added to corresponding sample.

TABLE 11
Effect of fermentation broth viscosity reduction upon enzyme
addition when compared to reference (no enzyme addition).

| Enzyme added | Viscosity reduction |
|---|---|
| Reference | 0 |
| SEQ ID NO: 420 | + |
| SEQ ID NO: 421 | + |
| SEQ ID NO: 422 | + |
| SEQ ID NO: 423 | + |
| SEQ ID NO: 424 | + |
| SEQ ID NO: 425 | + |

### Example 13. Enzyme Induced Aspergillus oryzae Culture Broth Viscosity Reduction

Fed batch fermentation on glucose employing a feeding strategy based on online feedback of dissolved oxygen level. Samples for offline viscosity measurements were treated with corresponding enzyme (Table 12) followed by incubation for 3 hours at 34° C. Viscosity reduction effect was normalized to protein concentration.

TABLE 12
Effect of fermentation broth viscosity reduction upon enzyme
addition when compared to reference (no enzyme addition).

| Enzyme added | Viscosity reduction |
|---|---|
| Reference | 0 |
| SEQ ID NO: 426 | + |
| SEQ ID NO: 427 | + |
| SEQ ID NO: 428 | + |
| SEQ ID NO: 429 | + |
| SEQ ID NO: 430 | + |
| SEQ ID NO: 431 | + |
| SEQ ID NO: 432 | + |
| SEQ ID NO: 433 (Mutanase) | + |

### Example 13. Bacillus Fermentation Broth Viscosity Reduction Using Diverse Muramidases and Off-Line Viscosity Measurements

A Bacillus licheniformis strain producing an amylase product was used to investigate the effect of enzyme addition to a culture broth. Viscosity of the culture broth was measured off-line following the addition of individual enzymes (Table 13).

TABLE 13
Fermentation broth viscosity reduction with no enzyme
addition (reference) compared to the addition of
diverse glycosyl hydrolases as indicated.

| Enzyme added | Viscosity reduction |
|---|---|
| Reference | 0 |
| SEQ ID NO: 434 | + |
| SEQ ID NO: 435 | + |
| SEQ ID NO: 436 | + |
| SEQ ID NO: 437 | + |
| SEQ ID NO: 438 | + |
| SEQ ID NO: 439 | + |
| SEQ ID NO: 440 | + |
| SEQ ID NO: 441 | + |
| SEQ ID NO: 442 | + |
| SEQ ID NO: 443 | + |
| SEQ ID NO: 444 | + |
| SEQ ID NO: 445 | + |
| SEQ ID NO: 446 | + |
| SEQ ID NO: 447 | + |
| SEQ ID NO: 448 | + |
| SEQ ID NO: 449 | + |
| SEQ ID NO: 450 | + |
| SEQ ID NO: 451 | + |
| SEQ ID NO: 452 | + |
| SEQ ID NO: 453 | + |
| SEQ ID NO: 453 | + |

1-16. (canceled)

17. A submerged fermentation process of producing a polypeptide of interest, the process comprising:
a) fermenting a microorganism that produces the polypeptide of interest in a fermentation broth, and
b) adding at least one enzyme to the fermentation broth, or co-expressing the at least one enzyme in the microorganism and secreting the at least one enzyme into the fermentation broth, in an amount sufficient to reduce the viscosity of the fermentation broth compared to when the at least one enzyme is not added or co-expressed.

18. The process of claim 17, further comprising recovering the polypeptide of interest.

19. The process of claim 17, wherein the polypeptide of interest is a mixture of two or more different polypeptides.

20. The process of claim 17, wherein the polypeptide of interest is one or more enzymes.

21. The process of claim 17, wherein the polypeptide of interest is selected from a hydrolase, isomerase, ligase, lyase, oxidoreductase, and transferase.

22. The process of claim 17, wherein the at least one enzyme is added as a bolus addition.

23. The process of claim 17, wherein the at least one enzyme is added as a continuous addition.

24. The process of claim 17, wherein the process is a fed batch fermentation process, and wherein the at least one enzyme is added in the feed.

25. The process of claim 17, wherein the process is a continuously fed fermentation process, and wherein the at least one enzyme is added in the feed.

26. The process of claim 17, wherein the at least one enzyme comprises at least one enzyme from a glycosyl hydrolase family.

27. The process of claim 17, wherein the at least one enzyme comprises at least one enzyme selected from a muramidase, chitinase, glucanase, mannanase, nuclease and phosphodiesterase.

28. The process of claim 17, wherein the at least one enzyme comprises a GH24 or GH25 muramidase comprising an amino acid sequence having at least 80% sequence identity to the amino acid sequence shown in any of SEQ ID NO: 1 to SEQ ID NO: 453, or a mature version thereof.

29. The process of claim 17, wherein the process comprises adding up to 20,000 mg of the at least one enzyme per kg of fermentation broth.

30. The process of claim 17, wherein the process comprises adding up to 500 mg/kg of the at least one enzyme per kg of fermentation broth.

31. The process of claim 17, wherein the process comprises adding the at least one enzyme in an activity ranging from below the detection limit of the muramidase assay and up to an activity of 0,200 limit of the muramidase assay when diluted 4,000 times.

32. The process of claim 17, wherein the process comprises adding the at least one enzyme in an activity ranging from below the detection limit of the muramidase assay and up to an activity of 0,200 limit of the muramidase assay when diluted 100 times.

33. The process of claim 17, wherein the at least one enzyme comprises a glycosyl hydrolase and a phosphodiesterase.

34. The process of claim 17, wherein the microorganism is bacterial.

35. The process of claim 34, wherein the microorganism is a Bacillus species.

36. The process of claim 34, wherein the microorganism is a Lactobacillus species.

37. The process of claim 17, wherein the microorganism is eukaryotic.

38. The process of claim 37, wherein the microorganism is an Aspergillus, Penicillium or Trichoderma species.

39. The process of claim 17, wherein the at least one enzyme is added to the fermentation broth by co-cultivating a second microorganism in the fermentation broth, wherein the second microorganism produces and secretes the at least one enzyme into the fermentation broth.

40. The process of claim 17, wherein the oxygen transfer rate is increased by increasing the agitation, stirring speed, mixing or oxygenation of the fermentation broth during the fermentation.
